(19) United States
(12) Patent Application Publication
(10) Pub. No.: US 2015/0164734 A1
(43) Pub. Date: Jun. 18, 2015

(54) System and Method for Cosmetic Enhancement of Lips

(71) Applicant: Guided Therapy Systems, LLC, Mesa, AZ (US)

(72) Inventors: Michael H. Slayton, Tempe, AZ (US); Peter G. Barthe, Phoenix, AZ (US)

(73) Assignee: Guided Therapy Systems, LLC, Mesa, AZ (US)

(21) Appl. No.: 14/568,954

(22) Filed: Dec. 12, 2014

Related U.S. Application Data
(60) Provisional application No. 61/915,485, filed on Dec. 12, 2013.

(51) Int. Cl. A61H 15/00; A61B 18/00

(52) U.S. Cl. CPC A61H 15/0092; A61B 18/00

(57) ABSTRACT

This disclosure provides systems and methods for cosmetic enhancement of lips. The systems and methods can include a treatment device including an energy source, a coupling material, a control module, and a housing. The systems and methods can generate and deliver an energy to the region of interest non-invasively to drive a cosmetic process about the lip or a physiological effect in the lip. The systems and methods can control the generation of the energy based on at least one of the physiological effect in the lip, a duration of generating the energy, a user input, and a temperature associated with the region of interest.

### CROSS-REFERENCE TO RELATED APPLICATIONS

This application is based on, claims priority to, and incorporates herein by reference for all purposes, U.S. Provisional Patent Application No. 61/915,485, filed Dec. 12, 2013.

### BACKGROUND

The art of enhancing the color and fullness of lips to create a pouty, sensual look, known in the art as “lip plumping”, has become increasingly popular, particularly among women. The popularity of full lips stems from a number of sources. For example, voluptuous lips have long been a sign of youth, beauty, fertility, and sensuality. Many believe that adding fullness and color to lips can make a face look younger.

It is also known to attempt to enhance lip size through topical application of a wide variety of compositions, for example, collagen may be topically applied to the lips. Compositions that enhance the size of blood vessels in the lips may be topically applied to the lips to cause swelling of the lips, such as for example, niacin, B vitamins, African chilies, hyacinth, or jasmine. All of these methods to enhance lips have drawbacks; therefore new approaches for the enhancement of lips are needed.

### SUMMARY

The present disclosure overcomes the aforementioned drawbacks by presenting systems and methods for cosmetic enhancement of lips.

In one aspect, this disclosure provides a treatment device for delivering an energy into a region of interest within a lip of a user. The treatment device can include an energy source, a coupling material, a control module, and a housing. The energy source can be configured to deliver an energy to the region of interest non-invasively to drive a cosmetic process about the lip. The coupling material can be arranged between the energy source and the lip to allow the energy to be transmitted from the energy source into the region of interest. The control module can be configured to control the energy source. The housing can contain the energy source and can be configured to be held by the user to arrange the energy source proximate to the lip to deliver the energy to the region of interest.

In another aspect, this disclosure provides a method for performing a non-invasive cosmetic process to a lip of a user. The method can include one or more of the following steps: generating an energy, using an energy source of a cosmetic device; directing the energy into a region of interest within the lip of the user to cause a physiological effect in the lip; and controlling the generating of the energy based on at least one of the physiological effect in the lip, a duration of generating the energy, a user input, and a temperature associated with the region of interest.

The foregoing and other aspects and advantages of the disclosure will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a side view illustrating an exemplary treatment device, according to one aspect of the present disclosure.

FIG. 2 is a side view illustrating an exemplary treatment device, according to one aspect of the present disclosure.

FIG. 3 is a side view illustrating an exemplary treatment device, according to one aspect of the present disclosure.

FIG. 4 is a side view illustrating an exemplary treatment device, according to one aspect of the present disclosure.

FIG. 5 is a side view illustrating an exemplary treatment device, according to one aspect of the present disclosure.

FIG. 6 is a side view illustrating an exemplary treatment device, according to one aspect of the present disclosure.

FIG. 7 is a top view illustrating an exemplary treatment device, according to one aspect of the present disclosure.

FIG. 8 is a top view illustrating an exemplary transducer, according to one aspect of the present disclosure.

FIG. 9 is a cross-sectional view along the line G-G of FIG. 8, according to one aspect of the present disclosure.

FIG. 10 is a side view illustrating an exemplary treatment device, according to one aspect of the present disclosure.

FIG. 11 is a side view illustrating an exemplary treatment device located between and in contact with two lips, according to one aspect of the present disclosure.

FIG. 12 is a first cross-sectional view along the line A-A of FIG. 10, according to one aspect of the present disclosure.

FIG. 13 is a second cross-sectional view along the line A-A of FIG. 10, according to one aspect of the present disclosure.

FIG. 14 is a first cross-sectional view along the line B-B of FIG. 10, according to one aspect of the present disclosure.

FIG. 15 is a cross-sectional view along the line C-C of FIG. 10, according to one aspect of the present disclosure.

FIG. 16 is a second cross-sectional view along the line B-B of FIG. 10, according to one aspect of the present disclosure.

FIG. 17 is a side view illustrating an exemplary treatment device, according to one aspect of the present disclosure.

FIG. 18 is a cross sectional view along the line D-D of FIG. 17, according to one aspect of the present disclosure.

FIG. 19 is a side view illustrating an exemplary treatment device, according to one aspect of the present disclosure.

FIG. 20 is a cross-sectional view along the line E-E of FIG. 19, according to one aspect of the present disclosure.

FIG. 21 is a cross-sectional view along the line F-F of FIG. 19, according to one aspect of the present disclosure.

FIG. 22 is a block diagram representative of an exemplary treatment device, according to one aspect of the present disclosure.

FIG. 23 is a side view illustrating the an exemplary treatment device in proximity to a user's lips, according to one aspect of the present disclosure.

FIG. 24 is a schematic representation of a cross-sectional view of the human lip.

FIG. 25 is a cross-sectional view illustrating an exemplary treatment device in contact with a lip, according to one aspect of the present disclosure.

FIG. 26 is a flowchart illustrating exemplary methods, according to one aspect of the present disclosure.

FIG. 27A is a schematic of an example of focused energy.

FIG. 27B is a schematic of an example of defocused energy.

FIG. 27C is a schematic of an example of weakly-focused energy.

FIG. 27D is a schematic of an example of unfocused energy.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices, and methods relating to improved ultrasound treatment efficiency and operation are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements.

The various embodiments may be described herein in terms of various functional components and processing steps. It should be appreciated that such components and steps may be realized by any number of hardware components configured to perform the specified functions. For example, various embodiments may employ various cosmetic enhancement devices, visual imaging and display devices, input terminals and the like, which may carry out a variety of functions under the control of one or more control systems or other control devices. In addition, the embodiments may be practiced in any number of cosmetic contexts and that the various embodiments relating to a method and system for acoustic tissue treatment as described herein are merely indicative of some of the examples of the applications for use in cosmetic enhancement. For example, the principles, features and methods discussed may be applied to any cosmetic application. Further, various aspects of the various embodiments may be suitably applied to cosmetic applications.

As used herein, the term cosmetic enhancement can refer to procedures, which are not medically necessary and are used to improve or change the appearance of a portion of the body. Since it is not medically indicated for improving one's physical well-being, cosmetic enhancement is typically an elective procedure. As used herein, cosmetic enhancement does not diagnose, prevent, treat, or cure a disease or other medical condition. Furthermore, cosmetic enhancement is not a method for treatment of the human or animal body by surgery or therapy and diagnostic methods practiced on the human or animal body. Cosmetic enhancement is a non-surgical and non-invasive procedure. In some embodiments, cosmetic enhancement can be a non-surgical and non-invasive procedure that is performed at home by a user who is not a medical professional.

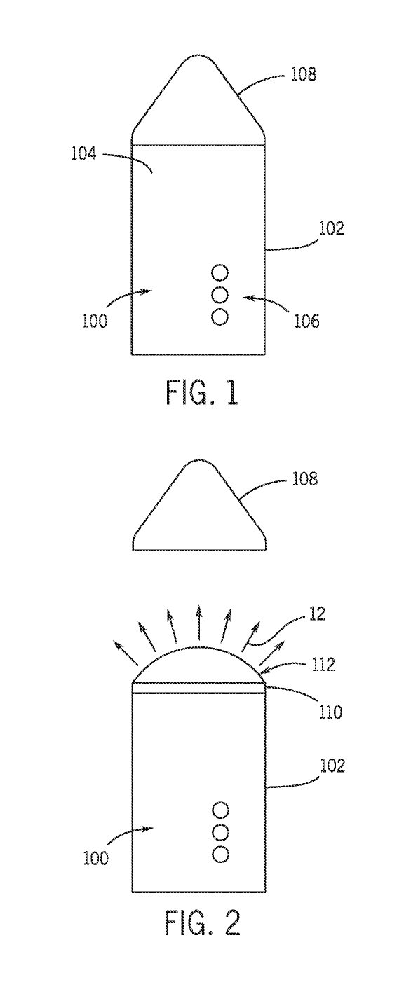

With reference to FIG. 1, a treatment device 100 is illustrated, in accordance with the present disclosure. The treatment device 100 can include a housing 102. The treatment device 100 can include a control module 104 and a display 106 coupled to or integrated with or within the housing 102. More particularly, the housing 102 can be configured to encase the control module 110. Also, the display 106 can be located on the housing 102. The treatment device can include a tip 108, which can be removable or disposable. FIG. 1 shows the tip 108 affixed to the rest of the treatment device 100.

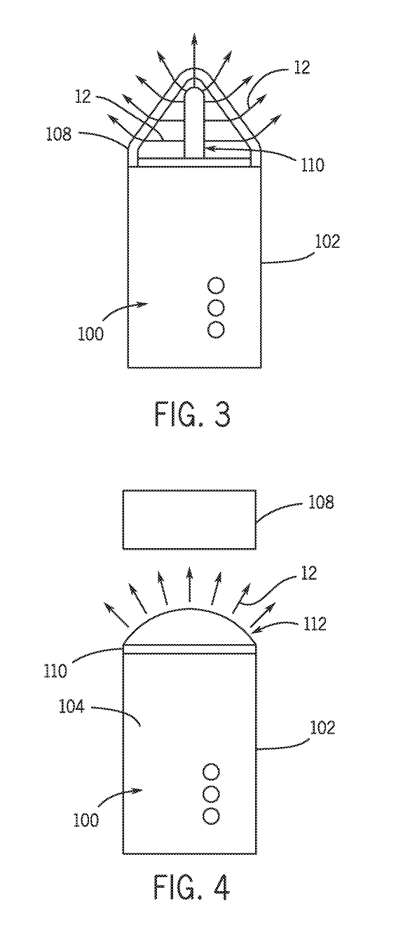

With reference to FIG. 2, the treatment device 100 is illustrated with the tip 108 removed from the rest of the treatment device 100. The treatment device 100 can include a transducer 110. The transducer 110 can emit energy 12. The treatment device 100 can also include a lens 112. The lens 112 can alter the trajectory of the energy 12, as desired for certain effects. As illustrated in FIG. 3 and will be further described, the tip 108 can alter the trajectory of the energy 12 that is emitted from the transducer 110. In addition, as shown in FIG. 4, the treatment device 100 can include a lens 112 that can alter the trajectory of the energy 12, as desired for certain effects.

In some configurations, the tip 108 can be substantially rectangular, and may be of suitable length that it provides treatment to all or any portion of the length and/or breadth of the lips. Moreover, the transducer 110 may be configured to emit acoustic energy 12 in more than one direction. The transducer 110 may include different settings to allow alternating between providing treatment to the upper and lower lip. The transducer 110 may be configured to provide treatment to the upper and lower lip at different frequencies. Other non-limiting examples of the system 100 configured in a lipstick configuration are described hereafter. In such examples, the lens 112 can be configured as a roller ball, which can be configured to emulate the feel of traditional lipstick. In some configurations, the roller ball can be configured with a coupling material which is applied to the lip 76 as the roller ball is moved. Coupling material can be any such materials, gels, medicants, as discussed herein, or are known to those skilled in the art now or at any time in the future.

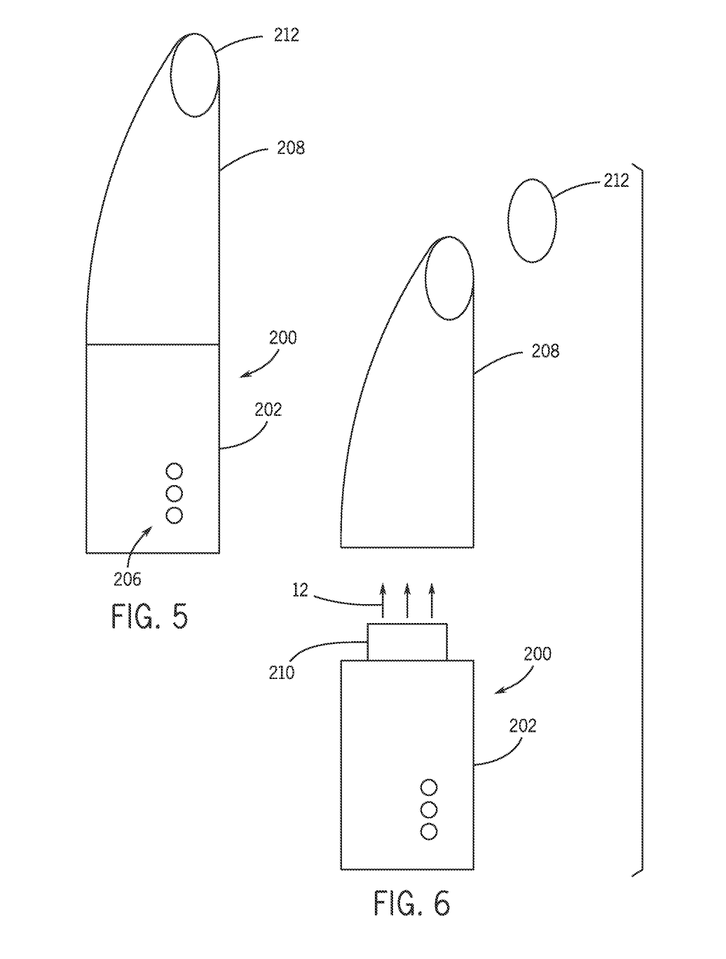

With reference to FIG. 5, a treatment device 200 is illustrated, in accordance with the present disclosure. The treatment device 200 can include a housing 202. The treatment device can include a display 206. The display can be located on or integrated with the housing 202. The treatment device 200 can include a tip 208. The treatment device 200 can include a lens 212. The lens 212 can be affixed to the tip 208. FIG. 6 shows the tip 208 and the lens 212 separated from one another and removed from the rest of the treatment device 200. As also illustrated in FIG. 6, the treatment device 200 can include a transducer 210. The transducer 210 can emit energy 12.

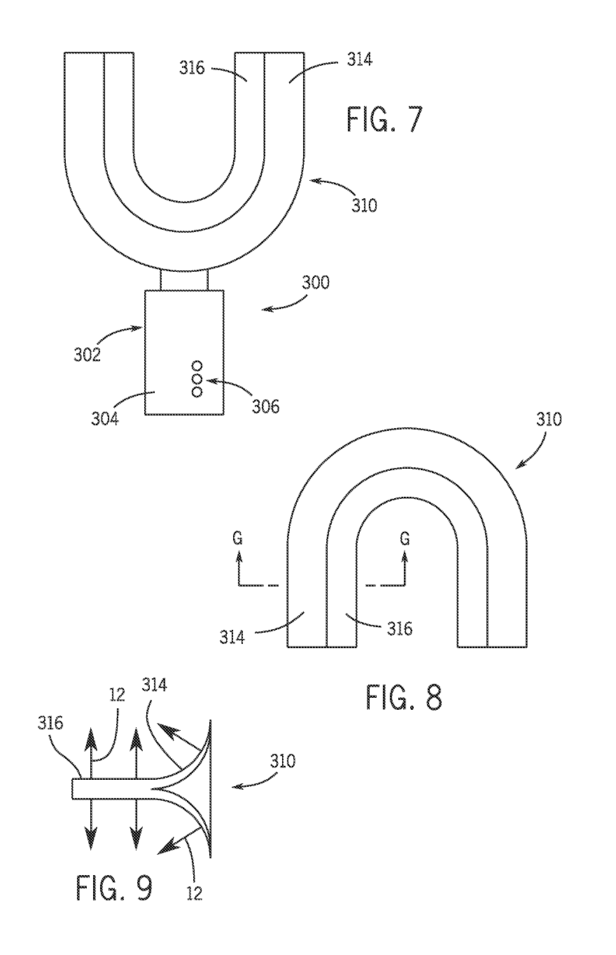

With reference to FIG. 7, a treatment device 300 is illustrated, in accordance with the present disclosure. The treatment device 300 can include a housing 302. The treatment device 300 can also include a control module 304. The housing 302 can be configured to encompass the control module 304. The treatment device 300 can include a display 306. The display 306 can be located on or integrated with the housing 302. The treatment device 300 can include a transducer 310 having a first portion 314 and a second portion 316. As illustrated in FIGS. 7 and 8, the transducer 310 can have a U-shape, for example, to treat all or substantially all of a users lip at one time.

More particularly, with reference to FIG. 9, a cross-section of the transducer 310 of FIGS. 7 and 8. Specifically, the cross-section of FIG. 9 is taken along line G-G of FIG. 8. As described, the transducer 310 includes a first portion 314 and a second portion 316. The first portion 314 can be curved. The second portion 316 can be substantially planar. The transducer 310 can emit energy 12. The energy 12 can be emitted from the first portion 314 and the second portion 316 in any order such as simultaneously, alternating, bidirectionally, or combinations thereof. In some configurations, a multidirectional transducer may be configured to function in the U-shaped configuration. In some embodiments of system 100, the housing and/or transducer can fold and/or collapse for compactness when not in use.

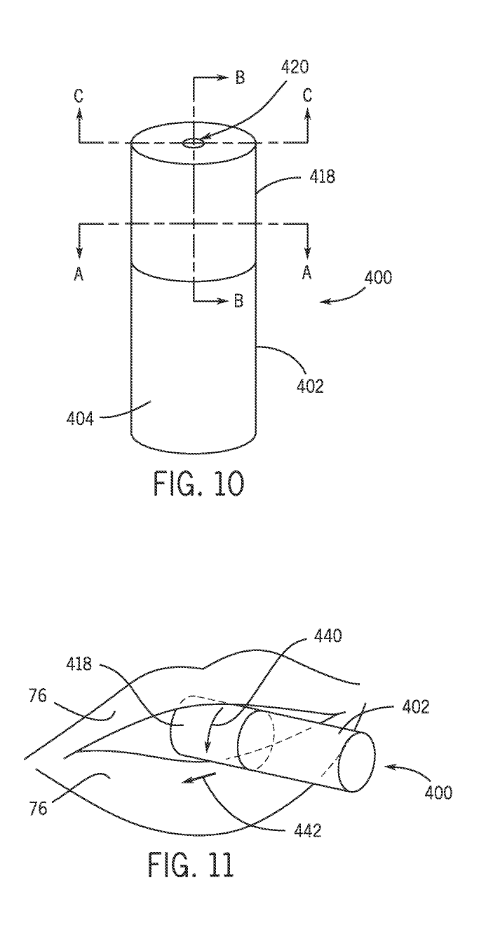

With reference to FIG. 10, a treatment device 400 is illustrated, in accordance with the present disclosure. The treatment device 400 can include a body 402. The treatment device 400 can include a control module 404. The body 402 can be configured to encompass the control module 404. The treatment device 400 can include a roller transducer assembly 418. The treatment device 400 can include an axle 420. The axle 420 can be configured to retain the roller transducer assembly 418 in its position and allow the roller transducer assembly 418 to rotate about the axle 420. The roller transducer assembly 418 can have a substantially cylindrical shape.

As will be described with respect to FIGS. 11-16, the treatment device 400 of FIG. 10 can be designed to include or be coupled with a variety of internal components. For example, with reference to FIG. 11, the roller transducer assembly 418 of the treatment device 400 can be configured to contact one or more lips 76 of a person. As the treatment device 400 is moved in a first direction 442 and contact is retained with the lip 76 illustrated beneath the treatment device 400, the roller transducer 418 can rotate in a rotational direction 440 relative to the body 402.

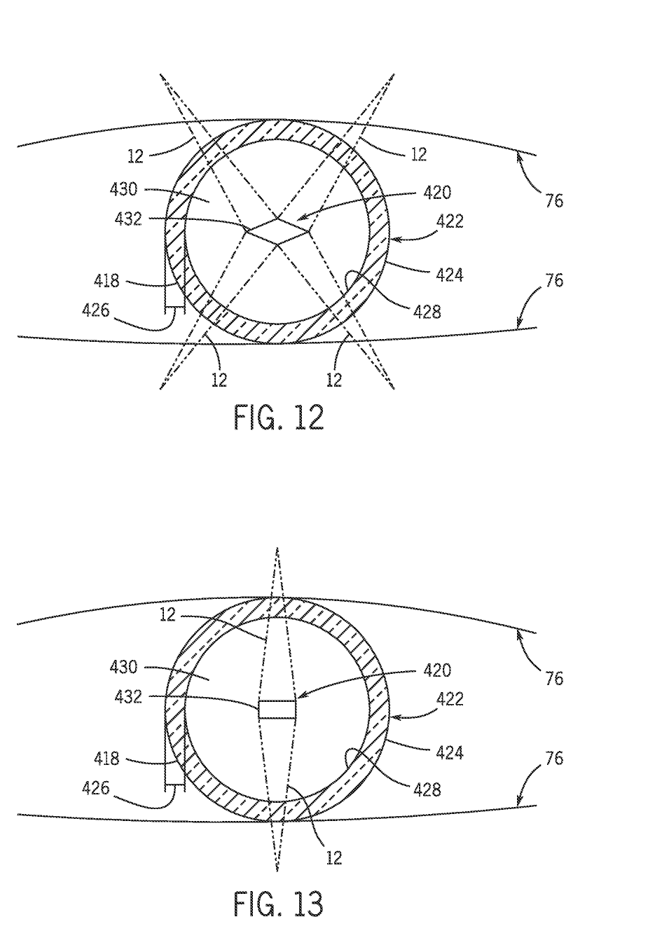

As another example, with reference to FIG. 12, a first cross-sectional view of the roller transducer assembly 418 of FIG. 10 is shown along the line A-A of FIG. 10. The roller transducer assembly 418 can include a transducer assembly body 422. The transducer assembly body 422 can have an outer surface 424, an inner surface 428, and a thickness 426, which is a distance measured from the outer surface 424 to the inner surface 428. The roller transducer assembly 418 can include an internal volume 430, which can be bounded by the inner surface 428. The roller transducer assembly 418 can include an axle 420 and an energy source 432. The axle 420 can be located at substantially a center point of the transducer assembly body 422. The energy source 432 can be located as substantially a center point of the transducer assembly body 422. The axle 420 and the energy source 432 can be a part of the same structure or separate structures. The energy source 432 can be located near the axle 420. The energy source 432 can be configured to emit energy 12. The energy source 432 can emit energy 12 in multiple directions, such as four directions as illustrated in the non-limiting example shown in FIG. 12. The energy source 432 can be a multi-directional transducer, which can facilitate treatment of both an upper and lower lip 76 in a coordinated fashion, including simultaneously, as a non-limiting example.

As yet another example, with reference to FIG. 13, a second cross-sectional view of the roller transducer assembly 418 of FIG. 10 is shown along the line A-A of FIG. 10. In this configuration, the energy source 432 can emit energy 12 in multiple directions, such as two directions as illustrated by the non-limiting example in FIG. 13. To this end, the energy source 432 can be a bi-directional transducer, which can facilitate treatment of both an upper and lower lip 76 in a coordinated fashion, including simultaneously, as a non-limiting example.

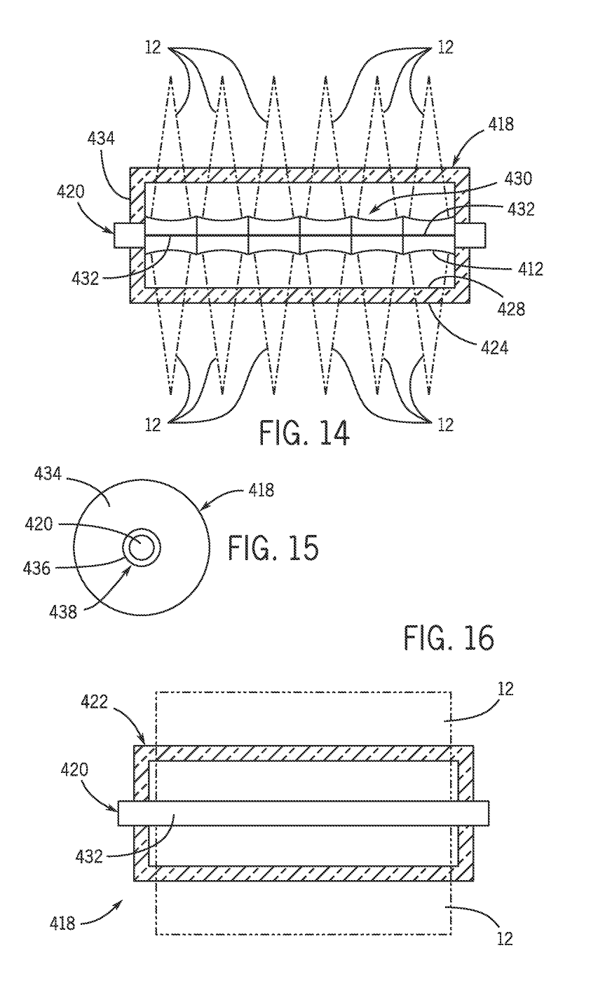

As still another example, with reference to FIG. 14, a first cross-sectional view of the roller transducer assembly 418 of FIG. 10 is illustrated along the line B-B of FIG. 10. In this configuration, the roller transducer assembly 418 can include a transducer assembly body 422 that, as will be described, can coordinate the deliver of energy 12 to a variety of locations. The transducer assembly body 422 can have an outer surface 424, an inner surface 428, and a thickness 426 which is a distance measured from the outer surface 424 to the inner surface 428. The transducer assembly body 422 can further have an end cap 434. The roller transducer assembly 418 can include an internal volume 430, which can be bounded by the inner surface 428 and the end cap 434. The roller transducer assembly 418 can include an axle 420 and an energy source 432. The axle 420 can be located at substantially a center point of the transducer assembly body 422. The energy source 432 can be located as substantially a center point of the transducer assembly body 422. The axle 420 and the energy source 432 can be a part of the same structure or separate structures. The energy source 432 can be located near the axle 420. The roller transducer assembly 418 can include one or more lenses 112 to alter the trajectory of the energy 12. The energy source 432 can be a bi- or multi-directional transducer, which can facilitate treatment of both an upper and lower lip 76 in a coordinated fashion, including simultaneously. The energy 12 can be focused. With reference to FIG. 16, a second cross-sectional view of a roller transducer assembly 418 of FIG. 10 is shown, again taken along the line B-B of FIG. 10. In this configuration, the energy 12 can be unfocused.

As a further example, with reference to FIG. 15, a cross-sectional view of a roller transducer assembly 418 of FIG. 10 is shown along the line C-C of FIG. 10. In this configuration, the roller transducer assembly 418 can include an end cap 434, an axle 420, and a seal 436 for sealing between the end cap 434 and the axle 420. The roller transducer assembly 418 can include an axle interface 438, such as a scotch yoke, for interfacing the axle with a treatment device 400 or other structure to which the axle is affixed or about which the axle rotates.

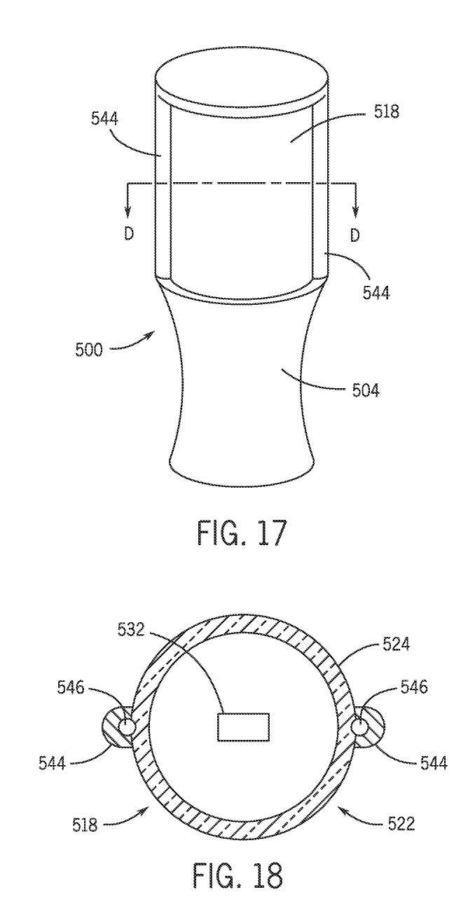

With reference to FIG. 17, a treatment device 500 is illustrated, in accordance with the present disclosure. The treatment device 500 can include a housing 502. The treatment device 500 can include a control module 504. The housing 502 can be configured to encompass the control module 504. The treatment device 500 can include a roller transducer assembly 518 and a coupling medium application device 544. The coupling medium application device 544 can distribute a coupling medium to assist coupling between the treatment device 500 and a lip 76.

With reference to FIG. 18, a cross-sectional view of the roller transducer assembly 518 along the line D-D of FIG. 17 is illustrated, in accordance with the present disclosure. The roller transducer assembly 518 can include a transducer assembly body 522 having an outer surface 524 and a coupling medium application device 544. The transducer assembly body 522 can rotate relative to the coupling medium application device 544. The coupling medium application device 544 can include an applicator 546 that places the coupling medium in contact with the outer surface 524. An energy source 432 can be located substantially at the center of the roller transducer assembly 518.

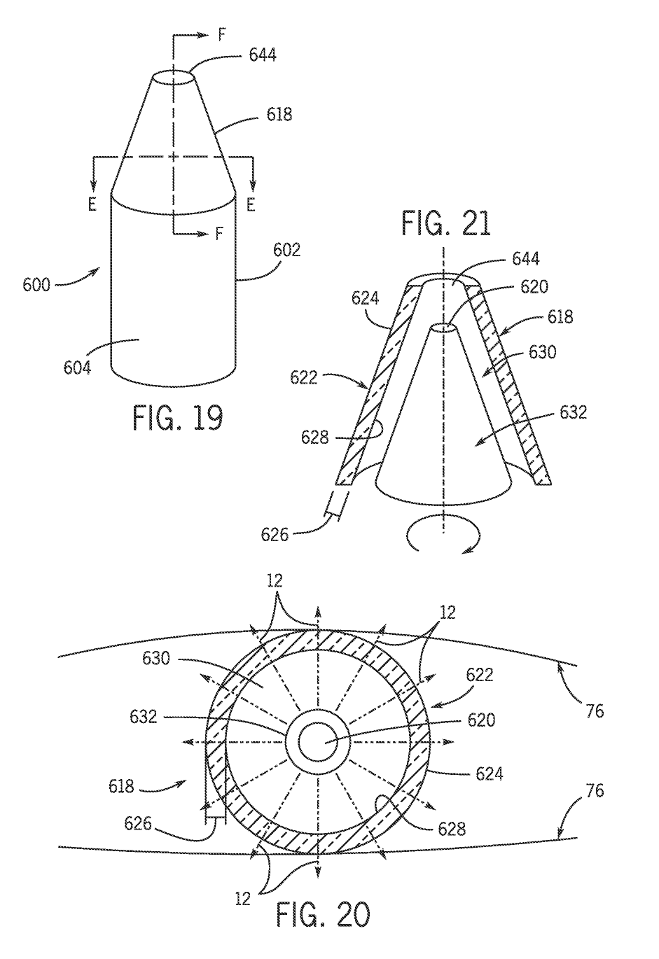

With reference to FIG. 19, a treatment device 600 is illustrated, in accordance with the present disclosure. The treatment device 600 can include a housing 602. The treatment device can include a control module 604. The housing 602 can be configured to encompass the control module. The treatment device 600 can include a roller transducer assembly 618 having a coupling medium application device 644. The roller transducer assembly 618 can have a substantially conical shape.

With reference to FIG. 20, a cross-sectional view of a roller transducer assembly 618 along the line E-E of FIG. 19 is illustrated, in accordance with the present disclosure. The roller transducer assembly 618 can include a transducer assembly body 622. The transducer assembly body 622 can have an outer surface 424, an inner surface 628, and a thickness 626 which is a distance measured from the outer surface 624 to the inner surface 628. The roller transducer assembly 618 can include an internal volume 630, which can be bounded by the inner surface 628. The roller transducer assembly 618 can include an axle 620 and an energy source 632. The axle 620 can be located at substantially a center point of the transducer assembly body 622. The energy source 632 can be located as substantially a center point of the transducer assembly body 622. The axle 620 and the energy source 632 can be a part of the same structure or separate structures. The energy source 632 can be located near the axle 620. The energy source 632 can be configured to emit energy 12. The energy source 632 can emit energy 12 in multiple directions. The energy source 632 can be a multi-directional transducer, which can facilitate treatment of both an upper and lower lip 76 in a coordinated fashion, including simultaneously. With reference to FIG. 21, the roller transducer assembly 618 of FIG. 19 is shown along the line F-F of FIG. 19. The axle 620 can include the energy source 63, which can be a multi-face transducer, such as described above, surrounding the axle 620.

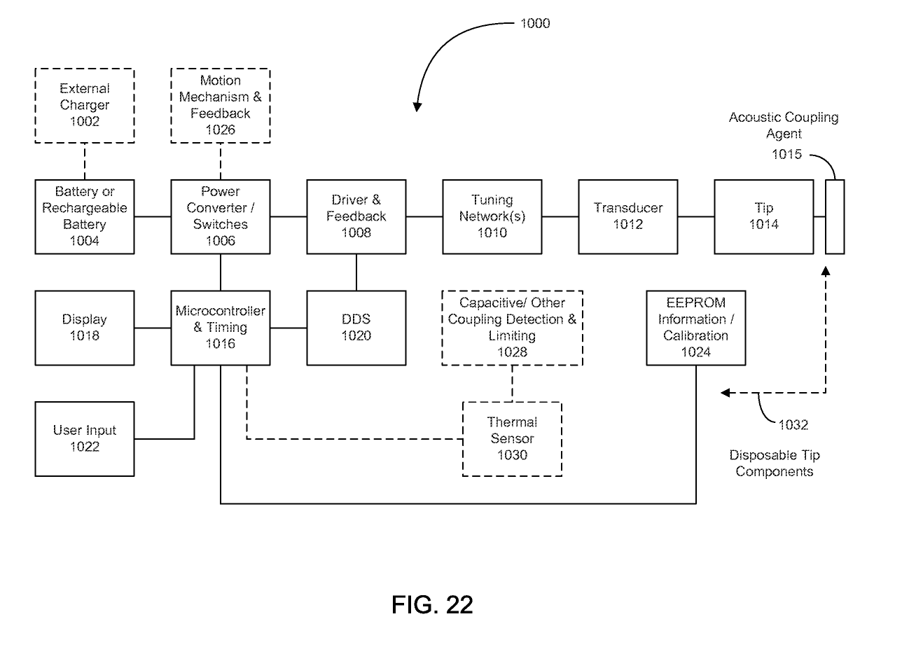

With reference to FIG. 22, a block diagram of a treatment device 1000 is illustrated as a block schematic diagram. The system 1000 may be controlled and operated by a hand-held format control system. As such, the system 1000 may include an external battery charger 1002 that can be used with rechargeable-type batteries 1004 or single-use disposable type batteries 1004, such as M-sized cells or AA cells, or the like. A power converter 1006 may be included to produce voltages for powering a driver/feedback circuit 1008 with tuning network 1010 for the driving a transducer 1012 that can deliver energy as described above.

As also described, the system 1000 may be configured to be coupled to the lip or lips of a user via one or more tips 1014, which can be composed of at least one of a solid media, semi-solid, such as, for example, a gelatinous media, and/or liquid media equivalent to an acoustic coupling agent contained within a housing in tip. The tip 1014 can be coupled to the lip with an acoustic coupling agent 1015. In some configurations, tip 1014 may include the transducer 1012. In such configurations, the tip 1014 and the transducer 1012 can be disposable and replaceable.

A controller and timing circuit 1016 may be included that carry out and coordinate a variety of processes and function. For example, the controller and timing circuit 1016 may be coupled to a display 1018. Also, the controller and timing circuit 1016 may direct digital synthesis (DDS) 1020 with associated software and algorithms to provide control and user interfacing via the display 1018, and/or LED type indicators, and other input/output controls, such as a user input 1022, such as switches and audio devices.

A storage element 1024, such as an electrically erasable programmable read-only memory (“EEPROM”), secure EEPROM, tamper-proof EEPROM, or similar device 1024 can hold calibration and usage data. A motion mechanism with feedback 1026 can be controlled to scan the transducer 1012 in a linear pattern or a two-dimensional pattern or over a varied depth. Other feedback controls may include capacitive, acoustic, or other coupling detection or limiting systems 1028, and a thermal sensor 1030. The EEPROM 1024 can be coupled with at least one of tip 1014, the transducer 1012, thermal sensor 1030, coupling or other limiting detectors 1028, or tuning network 1010 along with a plastic or other housing can form a disposable tip 1032.

In some configurations, sensing and monitoring components can include a sensor that is connected to an audio or visual alarm to prevent overuse of the transducer 1012. In some configurations, the sensor senses the amount of energy transferred to a ROI or the time that the transducer 1012 has be actively emitting energy. When a certain time or temperature threshold has been reached, the alarm sounds an audible alarm or causes a visual indicator to activate to alert the user that the threshold is reached. In some configurations, the sensor can be operatively connected to control system of system 1000 to force system 1000 to stop emitting ultrasound energy 7 from the transducer 1012.

The microcontroller 1016 has access to system software, such as may be stored in the EEPROM 1024, to control initialization, timing, level setting, monitoring, safety monitoring, and other system functions required to accomplish user-defined treatment objectives. Further, various control switches can also be configured to control operation. In some embodiments, system 1000 may also use the display system 1018 to provide images of the ROI. In certain configurations, the ultrasound energy is emitted from the transducer 1012 in a manner suitable for imaging. The display system 1018 can be configured to convey images or information apart from images about the transducer 1012 or the ROI to the user. Therefore, display system 1018 can be a computer monitor, touch or other screen or it can be a simply type of indicator system such a liquid crystal display or light emitting diode or organic light emitting diode display in various embodiments. Liquid crystal displays and light emitting diode displays and organic light emitting diode can be particularly useful in the display system 1018 when the transducer 1012 is a hand-held system.

In some configurations, data from the EEPROM 1024 can be downloaded to a user's computer via any interface type, such as, for example, a USB interface, a RS interface, a IEEE interface, a fire-wire interface, a blue tooth interface, an infrared interface, a 802.1 interface, the web, and the like. Downloadable data can include hours of use, frequency during use, power levels, depths, codes from tips used, error codes, user ID, and other such data. The data can be parsed by user ID so more than one user can track user data. Similarly, EEPROM 1024 can be interfaced, using any of the methods or devices described herein, to a computer or the web to receive software updates. Still further, the EEPROM 1024 can be interfaced, using any of the methods or devices described herein, to a computer or the web for at least one of diagnosis, trouble shooting, service, repair, and combinations thereof.

As described, the system 100 can include a feedback or limit control 1026, detection and limiting components 1028 a thermal sensor 1030, and a user input 1022 that may be coupled to the microcontroller 1016 to provide a variety of functionality. In some configurations, the microcontroller 1016 can use feedback from the thermal sensor 1030 to control or limit operation. The system 1000 can be configured to provide a temperature versus time profile selected to provide heating in the ROI below a thermal dose sufficient for ablation or coagulation. In some configurations, the temperature versus time profile meets one of the following conditions:

1) the ROI temperature of at least 50° C. is provided for a heating duration selected so as to deliver a thermal dose not causing an ablation or coagulation but not exceeding either one minute at 50° C. or one second in the range over 56° C.;

2) the ROI temperature is rapidly raised to a first level of greater than 50° C., then the ultrasound energy is turned off until the target area temperature drops to a second level of 35° C.-49° C., then the second level is maintained for a heating duration of at least one second;

3) the ROI temperature is raised to a first level of greater than 40° C., then the ultrasound energy is turned off until the target area temperature drops below a second level which is less than the first level, wherein said process of raising and lowering temperature is cyclically repeated to provide a thermostat effect;

4) the ROI temperature is raised to a first level of greater than 40° C., then the ultrasound energy is turned off until the target area temperature drops below a second level which is less than the first level by a few degrees, and then the target area temperature is raised to a third level exceeding the first level by a few degrees before the power is turned off again;

5) the ROI temperature is raised to at least 50° C. and held for at least one second, then the ultrasound energy is turned off for at least one second, wherein said process of raising the temperature and turning off the ultrasound energy is cyclically repeated to provide a pulsed profile; and

6) the ROI temperature of at least 46° C. is provided for a heating duration selected so as to deliver a thermal dose not causing an ablation or coagulation but not exceeding fifteen minutes at 46° C. or one second in the range over 56° C.

In some configurations, the upper limit of the temperature profile may be 60° C. The ROI can have a depth of a range of 1 micron to 10 millimeters below the skin surface. The system 1000 can be configured to emit ultrasound energy to provide mechanical effects to the target area or ROI. The system 1000 can be configured to emit ultrasound energy for mechanical effects comprising one of cavitation or streaming. In some configurations, the mechanical effects do not cause ablation or coagulation in the ROI. The system 1000 can include a medicinal agent configured to couple the transducer 1012 to the ROI. The transducer 1012 can be configured to deliver ultrasound energy to drive or push a portion of the medicinal agent through the surface and into the ROI. The system 1000 can be configured to raise the temperature in the ROI in a range from 35° C. to 49° C. The system 1000 can be configured to raise the temperature in the ROI in a range a range from 35° C. to 60° C.

As described above, the system 1000 may be enclosed in a housing. Any such housings as described above may be designed for comfort and control while used in an operator's hand. The housing may also contain various electronics, such as, for example, EEPROM, interface connection, motion mechanisms, and/or RAM for holding programs, and combinations thereof. In addition, the housing can include the above-described display 1018, such as indicator system such a liquid crystal display or light emitting diode display or organic light emitting diode display to provide feedback to the user. The housing can also include various input/output controls 1022 such as switches and/or buttons configured to control the system 1000.

The transducer 1012 can include a piezoelectrically active material, such as lead zirconante titanate (PZT), or any other piezoelectrically active material, such as a piezoelectric ceramic, crystal, plastic, and/or composite materials, as well as lithium niobate, lead titanate, barium titanate, and/or lead metaniobate. In addition to, or instead of, a piezoelectrically active material, the transducer 1012 can include any other materials configured for generating radiation and/or acoustical energy. The transducer 1012 can also include one or more matching and/or backing areas configured along with transduction element such as coupled to the piezoelectrically active material. The transducer 1012 can also be configured with single or multiple damping elements along transduction element.

In accordance with some configurations, the thickness of transduction element of the transducer 1012 can be configured to be uniform. That is, transduction element can be configured to have a thickness that is substantially the same throughout. In accordance with some configurations, the transduction element can also be configured with a variable thickness, and/or as a multiple damped device. For example, the transduction element of the transducer 1012 can be configured to have a first thickness selected to provide a center operating frequency of a lower range, for example from approximately 1 kHz to 3 MHz. The transduction element can also be configured with a second thickness selected to provide a center operating frequency of a higher range, for example from approximately 3 to 100 MHz or more.

The transducer 1012 can be configured as a single broadband transducer excited with at least two or more frequencies to provide an adequate output for raising the temperature within ROI to the desired level. The transducer 1012 can also be configured as two or more individual transducers, wherein each the transducer 1012 includes a transduction element. The thickness of transduction elements can be configured to provide center-operating frequencies in a desired treatment range. For example, the transducer 1012 can comprise a first transducer 1012 configured with a first transduction element having a thickness corresponding to a center frequency range of approximately 1 MHz to 3 MHz, and a second transducer 1012 configured with a second transduction element having a thickness corresponding to a center frequency of approximately 3 MHz to 100 MHz or more. Various other ranges of thickness for a first and/or second transduction element can also be realized.

For example, as described above with respect to FIG. 12, the transducer assembly 418 can be configured to emit energy from at least two faces. For example, transducer assembly 418 can be configured to emit energy from four faces. Thus, the transducer assembly 418 may be configured to provide treatment to both an upper lip and a lower lip simultaneously. Thus, increasing the number of radiating surface areas can enable treatment over a larger ROI. Accordingly, as described above with respect to FIG. 14 additional faces of the transducer assembly can facilitate treatment over a given ROI in less treatment time and use less output power. In some embodiments multiple emitting faces can be implemented via suitably disposed multiples of transducer.

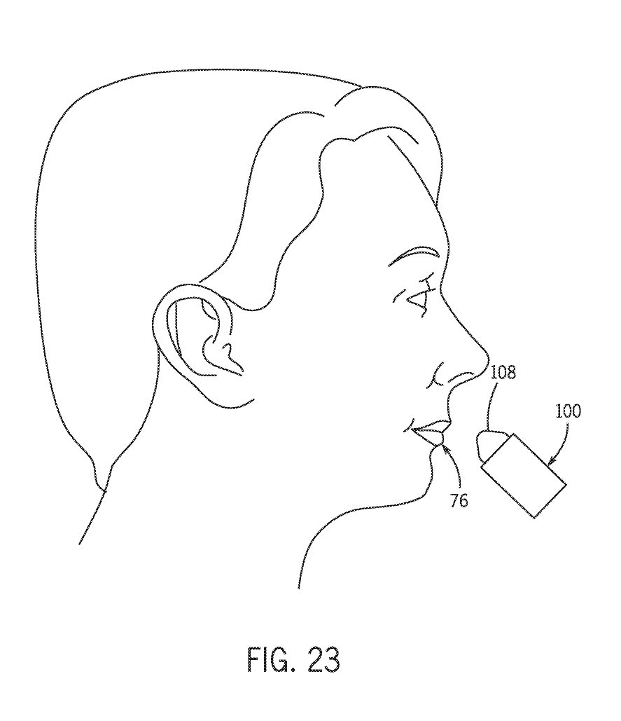

With reference to FIG. 23, the treatment device 100, or other configurations of treatment devices, may be positioned in proximity to a user's lips 76. To that end, the tip 108, or other configurations of tips as described above, can be adapted for coupling the treatment device 100 to the user's lips 76. As illustrated, the device 100 may be hand-held device formed similarly to a lipstick tube configuration. For example, tip 108 of hand held device 100 may be configured in a shape of any known lipstick product to facilitate application to the lips 76. In some configurations, the tip 108 is configured such that it may be pursed between lips 76. However, it will be appreciated that a hand-held device 100 may be formed in other configurations that are suitable for particular purposes or applications. In accordance with some configurations, the tip 108 may form an upside-down “V” configuration, such that it may be pursed between the user's lips.

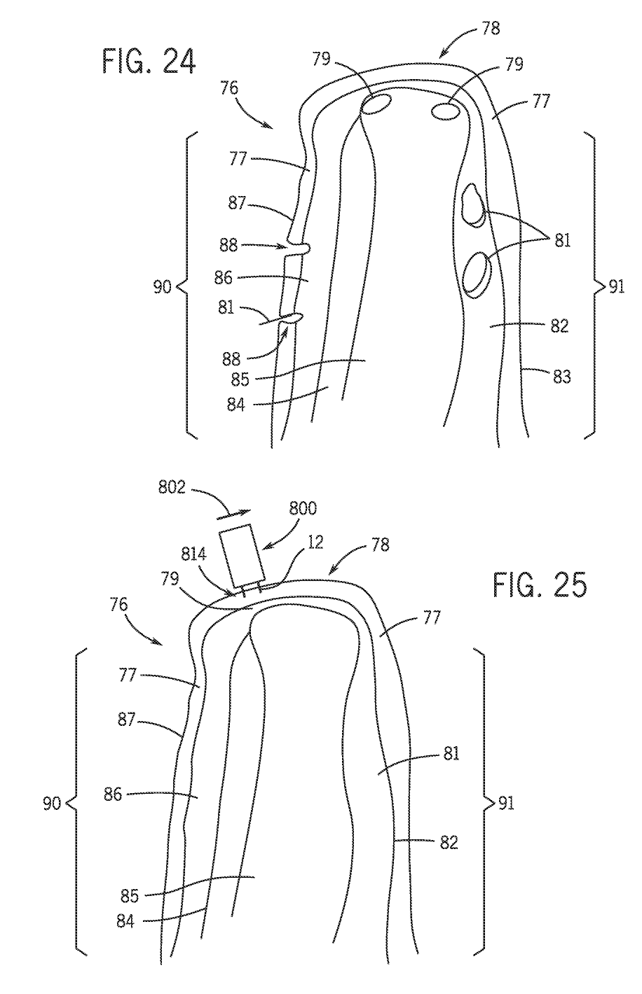

With reference to FIG. 24, a schematic representation of a cross-sectional view of the human lip 76 is illustrated, in accordance with the present disclosure. The human lip 76 can include a vermilion 77, a vermilion surface 78, a blood vessel 79, a salivary gland 81, a lamia proria 82, a mucosa 83, subcutaneous tissue 64, an orbicularis oris muscle 85, a dermis 86, an epidermis 87, a sebaceous gland 88, and hair 89. The human lip 76 can include a cutaneous lip 90 and a mucosal lip 91.

With reference to FIG. 25, a treatment device 800 in contact with a schematic representation of a lip 76 is illustrated, in accordance with the present disclosure. The treatment device 800 can be coupled to the vermillion surface 78 and transmits energy 12 into the vermillion 77.

The system 800 can be configured to treat the ROI 814. The system 800 can moved in a forward direction 802, which can move or rotate the transducer of the system 800, such as described above, along surface of lip 76 and treat a larger ROI 814. However, the system 800 can moved in a motion that is opposite forward motion 802, which will move or rotate the transducer of the system 800 in the opposite direction. Furthermore, the system 800 can be moved back and forth in any pattern that the user chooses. The system 800 can be configured with an acoustic gel application device, such as described above, which can apply a coupling material between the lip 76 and the system 800. The coupling material is applied to the surface of the lip 76 during movement or rotation of the system 800 across the lip 76. The coupling material can be any such materials, gels, medicants, as discussed herein, or are known to those skilled in the art now or at any time in the future. The coupling material can be a lipstick composition. In such configurations, as will be described, the transducer assembly body may be slightly porous assembly which allows for the lipstick material to be delivered to the surface of the lip 76 when the transducer is emitting energy 12. In such embodiments, the transducer assembly may be disposable so that a new transducer assembly can be purchased to replace the used transducer. In some configurations, the transducer assembly 150 is interchangeable, which can allow a user to choose a color from a library of available lipstick colors.

In some configurations, the acoustic gel application device may be configured to accept a conventional lipstick case and apply the lipstick within the case to the transducer assembly. In some configurations, the acoustic gel application device includes a bias member, which is employed to push the coupling material onto transducer assembly and/or keep the coupling material in contact with the outer surface of the transducer assembly or lip. In some embodiments, the acoustic gel application device is configured to dispense the coupling material in the form of a gel lipstick. In such embodiments, user can apply the coupling material like traditional lipstick then treat the lips. The coupling material 107 can be any such materials, gels, medicants, as discussed herein, or are known to those skilled in the art now or at any time in the future.

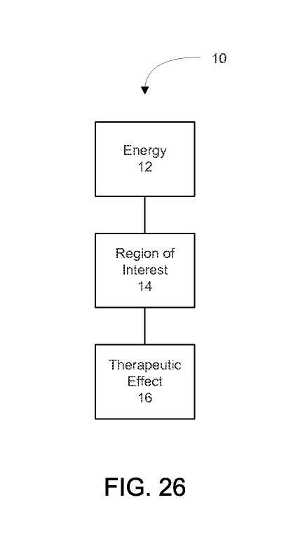

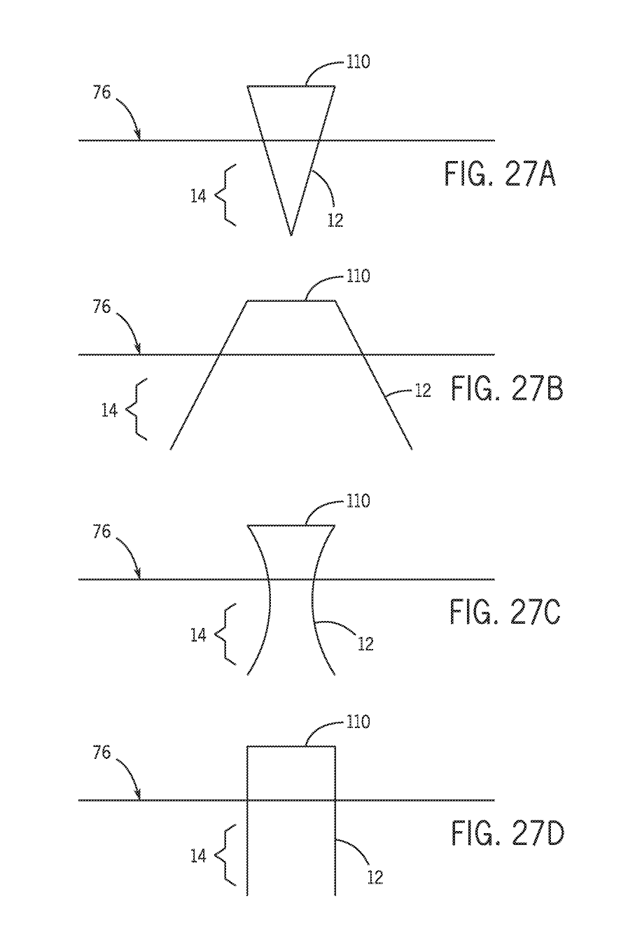

With reference to FIG. 26, a flowchart illustrating a method 10 is illustrated, in accordance with the present disclosure. The method can include directing an energy 12 into a region of interest 14 thereby causing a physiological effect 16.

The methods and systems described herein can be non-invasive. The methods and systems described herein can direct controlled energy, such as ultrasound energy, into at least one layer of tissue in a lip to create a conformal region of elevated temperature in the tissue.

The treatment device 100, 200, 300, 400, 500, 600, 700, 800 can be a hand-held device. The treatment device 100, 200, 300, 400, 500, 600, 700, 800 can include a rechargeable power supply. The treatment device 100, 200, 300, 400, 500, 600, 700, 800 can be configured in a lipstick shape, where a transducer is located in a tip that can be pursed between a user's lips 76, a “U’- or “V”-shape, where a transducer can be pursed between a user's lips 76.

The transducer can emit energy in more than one direction. The transducer can be a bi-directional transducer, such as, for example, the bi-directional transducers described in U.S. Pat. No. 7,393,325, which is incorporated herein in its entirety by reference.

The tip can be attached to the surface of the lip by suction. A negative pressure differential can be created, which can attach the tip to the exterior surface of the lip. The suction can ensure that the energy is directed into the lip and can reduce a thickness of the tissue being treated, which can decrease treatment time. The tip can include or have applied to its surface a coupling agent, a medicament, a glossing agent, or a coloring agent.

The region of interest 14 can be at least one of located in a subcutaneous area, a orbicularis muscle area, or a mucous membrane area. The coupling medium can include a medicament, which upon delivery of energy to the lip can be delivered into the tissue of the lip. The medicament can be a skin moisturizer or an ultraviolet protector.

The energy 12 can be any one of microwave energy, radio frequency energy, photon-based energy, ultrasound energy, thermal energy (such as using a resistive heater), or a combination thereof. In certain applications, the energy 12 can be ultrasound energy. The energy 12 can be ablative or non-ablative. The energy 12 can create a conformal lesion in the region of interest 14.

The energy 12 can be unfocused ultrasound energy, defocused ultrasound energy, or focused ultrasound energy. Ultrasound energy 12 can be spatially, temporally, or spatially and temporally controlled at least partly by changing spatial parameters of a transducer, such as the placement, distance, treatment depth, and structure of the transducer, or by changing temporal parameters of the transducer, such as the frequency, drive amplitude, and timing.

The methods and systems described herein can be configured to heat a region of interest 14 from 1 mm to 10 mm below the surface of a lip 76 by application of energy, particularly ultrasound energy. In certain applications, the heating can be from 1 mm to 5 mm below the surface of the lip 76. In certain applications, the region of interest 14 can be located from 1 mm to 3 mm below the surface of the lip 76, greater than 5 mm below the surface of the lip 76, or from 7 mm to 10 mm below the surface of the lip 76.

The methods and systems described herein can cause thermal or mechanical effects in the region of interest 14. The methods and systems described herein can raise the temperature of a region of interest 14 from about 1° C. to about 25° C. or from about 1° C. to about 15° C. above a normal body temperature. The methods and systems described herein can raise the temperature of a region of interest 14 to a range from about 35° C. to about 60° C., from about 35° C. to about 49° C., from about 40° C. to about 55° C., from about 43° C. to about 48° C., from about 43° C. to about 45° C., or below a threshold of ablation of the tissue. The temperature can be raised without causing ablation or coagulation of the tissue. Heating at the region of interest 14 can cause better diffusion of medicaments within the region of interest 14.

In certain aspects, the temperature increase can be very high but for a short enough time period so as to not cause ablation or coagulation. Alternatively, the temperature increase can be small and applied long enough to cause an effect. The temperature in the region of interest 14 can be raised to 50° C. or more and held for several seconds, such as from one second to five seconds. This treatment can be applied in a pulsed fashion.

A time-temperature profile of the treatment can be modeled and controlled with the aid of the thermal dose concept. The thermal dose, or t₄₃, is the exposure time at 43° C., which causes an equivalent biological effect due to an arbitrary time-temperature heating profile. Typically an ablative lesion forms on the order of one second at 56° C., which corresponds to a thermal dose of one hundred and twenty minutes at 43° C. The same thermal dose corresponds to 50° C. for approximately one minute. Thus, a non-ablative profile can contain high temperatures for very short times and/or lower temperatures for longer times or a combination of various time-temperature profiles. For example, temperatures as high as 56° C. for under one second or 46° C. for under fifteen minutes can be utilized. Such processes can be implemented in various aspect, whereby one or more profiles may be combined into a single treatment.

In some aspects, the temperature can be raised quickly to a high level (greater than 50° C.), and then dropped to a lower temperature (less than 50° C.), and then maintained at that temperature for a given time period such as one second up to several seconds or over a minute.

In some aspects, the temperature can be increased quickly to a high level (T HIGH), whereby T HIGH is greater than 40° C., and the power to transducer is turned off, but turned on again once the temperature drops below a lower threshold, (T LOW), whereby T LOW is less than T HIGH. Once the temperature reaches T HIGH again power to transducer is turned back off and this process is repeated, in effect acting like a thermostat. The process is terminated after a total treatment time of less than one second to one minute or more.

Systems and methods described herein can be configured to provide a temperature versus time profile selected to provide heating in the ROI 14 below a thermal dose sufficient for ablation or coagulation. In some embodiments, the temperature versus time profile meets one of the following conditions:
1) the ROI 14 temperature of at least 50° C. is provided for a heating duration selected so as to deliver a thermal dose not causing an ablation or coagulation but not exceeding either one minute at 50° C. or one second in the range over 56° C.,
2) the ROI 14 temperature is rapidly raised to a first level of greater than 50° C., then the ultrasound energy is turned off until the target area temperature drops to a second level of 35° C.-49° C., then the second level is maintained for a heating duration of at least one second,
3) the ROI 14 temperature is raised to a first level of greater than 40° C., then the ultrasound energy is turned off until the target area temperature drops below a second level which is less than the first level, wherein said process of raising and lowering temperature is cyclically repeated to provide a thermostat effect,
4) the ROI 14 temperature is raised to a first level of greater than 40° C., then the ultrasound energy is turned off until the target area temperature drops below a second level which is less than the first level by a few degrees, and then the target area temperature is raised to a third level exceeding the first level by a few degrees before the power is turned off again,
5) the ROI 14 temperature is raised to at least 50° C. and held for at least one second, then the ultrasound energy is turned off for at least one second, wherein said process of raising the temperature and turning off the ultrasound energy is cyclically repeated to provide a pulsed profile,
6) the ROI 14 temperature of at least 46° C. is provided for a heating duration selected so as to deliver a thermal dose not causing an ablation or coagulation but not exceeding fifteen minutes at 46° C. or one second in the range over 56° C.

Mechanical effects can include cavitation, streaming, and sheer stress of cells within the lip 76. Mechanical effects can create various forces which can cause tissue affectation. Tissue affectation can cause increased blood perfusion, which can enhance the color of the lip 76 or increase the size of the lip 76. Mechanical effects can help drive medicinal creams, such as moisturizers, ultraviolet protectants, collagen, and other agents into cells to better effectuate treatment.

The methods and systems described herein can cause a physiological effect that can enhance lip size and color. The methods and systems described herein can cause perfusion of blood to capillary vessels in a lip, which can enhance color. The perfusion of blood can also result in dilation of capillary vessels, which can enhance size of the lips, thereby achieving a desirable cosmetic effect. The methods and systems can induce a transient edema in a portion of a lip. The methods and systems can stimulate various natural glands to keep lips moist, which can prevent or treat dry and cracked lips.

The systems and methods described herein can elevate the temperature at a depth below the surface of a lip 76, but can leave the temperature of any intervening tissue substantially unchanged.

The systems and methods described herein can include assisting in medicament delivery. Efficacy of certain medicaments can be increased by the application of ultrasound energy. Certain medicaments can be activated by the application of ultrasound energy.

In certain various configurations, the transducer or system may be equipped with certain features to aid the user. For example, as described, a disposable tip can covers the transducer during use. The disposable tip allows ultrasound energy to pass through the tip and contact the lip. But, the disposable tip can be removed from system after use and replaced with a new disposable tip to prevent the spread of germs from one user to another that might reside on the transducer after contact with the lip. Different size disposable tips can be used and fall within the scope of the present disclosure. Additionally, a cap or a lid may be provided to protect tip and keep it free of debris. In some configurations, the disposable tip may include various compositions. For example, the disposable tip may include a coupling agent, medicant, glossing agent, and/or a color-adding agent.

In accordance with some configurations, a monitoring method may include monitoring the temperature profile or other tissue parameters of the ROI, such as attenuation, speed of sound, or mechanical properties such as stiffness and strain of the treatment region and suitably adjust the spatial and/or temporal characteristics and energy levels of ultrasound energy emitted from the transducer. The results of such monitoring techniques may be indicated on display system, such as for example, LED diodes and/or LCD display, by a success or fail type indicator, or combinations thereof. Additional treatment monitoring techniques may be based on one or more of temperature, video, profilometry, and/or stiffness or strain gauges or any other suitable sensing technique.

The present invention has been described above with reference to various exemplary configurations. However, those skilled in the art will recognize that changes and modifications may be made to the exemplary configurations without departing from the scope of the present invention. For example, the various operational steps, as well as the components for carrying out the operational steps, may be implemented in alternate ways depending upon the particular application or in consideration of any number of cost functions associated with the operation of the system, e.g., various of the steps may be deleted, modified, or combined with other steps. Further, it should be noted that while the method and system for ultrasound treatment as described above is suitable for use by a medical practitioner proximate the patient, the system can also be accessed remotely, i.e., the medical practitioner can view through a remote display having imaging information transmitted in various manners of communication, such as by satellite/wireless or by wired connections such as IP or digital cable networks and the like, and can direct a local practitioner as to the suitable placement for the transducer. Moreover, while the various exemplary embodiments may comprise non-invasive configurations, system can also be configured for at least some level of invasive treatment application. These and other changes or modifications are intended to be included within the scope of the present invention, as set forth in the following claims.

1. A treatment device for delivering an energy into a region of interest within a lip of a user, the treatment device comprising:
an energy source configured to deliver an energy to the region of interest non-invasively to drive a cosmetic process about the lip;
a coupling material arranged between the energy source and the lip to allow the energy to be transmitted from the energy source into the region of interest;
a control module configured to control the energy source; and
a housing containing the energy source and configured to be held by the user to arrange the energy source proximate to the lip to deliver the energy to the region of interest.

2. The device of claim 1 further comprising at least one of a tip or a lens configured to engage the lip and allow the energy to pass therethrough to toward the region of interest.

3. The device of claim 2 wherein the at least one of the tip or the lens forms roller ball, a roller, a porous assembly, a conical-shape, a u-shape, or a v-shape.

4. The device of claim 2 wherein the at least one of the tip or the lens is configured to hold the coupling material for dispensing between the energy source and the lip.

5. The device of claim 4 wherein the coupling medium includes a coloring agent configured to remain on the lip of the user.

4. The device of claim 5 wherein the coupling medium forms at least one of a gel, gelatinous media, liquid media, acoustic coupling agent, a medicant, moisturizer, and/or ultraviolet protector.

5. The device of claim 1 wherein the energy source includes a transducer configured to emit the energy along an axis extending away from the transducer.

6. The device of claim 5 further comprising at least one of a tip and a lens arranged along the axis and configured to at least one of disperse or focus the energy prior to arriving at the region of interest.

7. The device of claim 5 wherein the axis extends in opposite directions away from the transducer to deliver the energy to two regions of interest located on opposite sides of the device.

8. The device of claim 7 wherein the transducer is configured to emit the energy along another axis offset from the axis extending away from the transducer to deliver the energy to more than two regions of interest.

9. The device of claim 7 wherein the control module is configured to cause the transducer to emit the energy according to a coordinated delivery plan to deliver the energy to the two regions of interest simultaneously.

10. The device of claim 1 wherein the energy source includes a plurality of transducers configured to emit the energy along an axis extending away from the transducer toward the region of interest.

11. The device of claim 1 wherein the energy source is configured to deliver the energy with properties configured to cause at least one of heating or mechanical effects in the region of interest.

12. The device of claim 11 wherein the mechanical effects include at least one of cavitation or streaming.

13. The device of claim 11 wherein the heating is performed relative to at least one of ablation or coagulation in the region of interest.

14. The device of claim 1 further comprising at least one sensor configured to monitor at least one of motion of the device, power consumed by the device, coupling of the device to the lip, or thermal effects.

15. The device of claim 14 wherein the control module is configured to at least one of coordinate or disrupt operation of the energy source based on feedback from the at least one sensor.

16. The device of claim 1 wherein the housing is configured to at least one of fold or collapse.

17. The device of claim 1 further comprising a display configured to at least one of provide feedback to a user of the device or receive user input.

18. The device of claim 1 wherein the energy includes at least one of microwave energy, radio frequency energy, photon-based energy, ultrasound energy, resistive heating energy, or a combination thereof.

19. A method for performing a non-invasive cosmetic process to a lip of a user, the method comprising:
a) generating an energy, using an energy source of a cosmetic device;
b) directing the energy into a region of interest within the lip of the user to cause a physiological effect in the lip; and
c) controlling the generating of the energy in step a) based on at least one of the physiological effect in the lip, a duration of generating the energy, a user input, and a temperature associated with the region of interest.

20. The method of claim 19 further comprising arranging a coupling material arranged between the device and the lip to allow the energy to be transmitted from the energy source into the region of interest and wherein the coupling medium forms at least one of a lip coloring agent, a gel, gelatinous media, liquid media, acoustic coupling agent, a medicant, moisturizer, and/or ultraviolet protector.
